(19) United States
(12) Patent Application Publication
(10) Pub. No.: US 2005/0106104 A1
(43) Pub. Date: May 19, 2005

(54) Methods for diagnosing cardiovascular disorders

(76) Inventor: Alexander F. Rosenberg, Brookline, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/944,924

(22) Filed: Sep. 20, 2004

Related U.S. Application Data
(60) Provisional application No. 60/504,264, filed on Sep. 18, 2003.

(51) Int. Cl. G01N 33/53; A61K 49/00

(52) U.S. Cl. 424/9.2; 435/7.1

(57) ABSTRACT

Disclosed are methods of detecting cardiovascular disorders using reference profiles. Also disclosed are methods of identifying agents for treating cardiovascular disorders.

### FIELD OF THE INVENTION

The invention relates to methods of diagnosing cardiovascular disorders.

### BACKGROUND OF THE INVENTION

Cardiovascular disorders (CVD), principally heart disease and stroke, are among the nation's leading killers for both men and women and among all racial and ethnic groups. More than 61 million Americans have some form of CVD, including high blood pressure, coronary heart disease, stroke, angina, congestive heart failure, hypertrophic cardiomyopathy, and other conditions. More than 2,600 Americans die each day of CVD. In the United States, economic damage caused by CVD, including health expenditures and lost productivity, exceeded $329.2 billion in 2002.

### SUMMARY OF THE INVENTION

The invention is based on the discovery of a profile of analytes that are correlated with cardiovascular disorders. Cardiovascular disorders include, for example, high blood pressure, coronary heart disease, unstable angina, stroke, and congestive heart failure. The analytes that are differentially present in cardiovascular disorders are referred to herein as “CVD-X,” or CVD-X analytes. These analytes, as well as metabolites, derivatives, homologs, analogs and fragments thereof, will hereinafter be collectively designated as CVD-X, where X is an integer between 1 and 112. A profile containing the relative levels of two or more CVD-X members is known as a cardiovascular disorder reference profile.

The invention provides a cardiovascular disorder reference profile that includes a pattern of two or more analytes or metabolites thereof of CVD 1-112. Alternatively, the invention provides a cardiovascular disorder reference profile that includes a pattern of two or more analytes or metabolites of CVD 1-25, CVD 26-39, CVD 40-54, CVD 55-74, CVD 75-98, or CVD 99-112.

The invention also provides methods of diagnosing a cardiovascular disorder (CVD), or a predisposition to developing a cardiovascular disorder in a subject by determining a level of a CVD-associated analyte in a patient derived sample. An alteration, e.g., an increase or a decrease of the level compared to a normal control level indicates that the subject suffers from or is at risk of developing a cardiovascular disorder.

By a “CVD-associated analyte” is meant an analyte that is characterized by being present at a level that differs in a biological sample obtained from an individual with a cardiovascular disorder or at risk of developing a cardiovascular disorder, compared to a control biological sample. A control biological sample includes a biological sample obtained from a normal (control) individual. A normal control individual is a healthy individual or population of individuals known not to be suffering from a cardiovascular disorder. For example, a control level is a database of patterns from previously tested individuals. A normal individual is one with no clinical symptoms of a cardiovascular disorder. Alternatively, a control biological sample includes a biological sample obtained from the individual with a cardiovascular disorder or at risk of developing a cardiovascular disorder taken at a time prior to the onset of the cardiovascular disorder. A CVD-associated analyte is one or more of CVD 1-112.

The level of the analyte is increased 1.1-fold, 1.25-fold, 1.5-fold, 1.75-fold, 2-fold, 5-fold, 10-fold, 25-fold, 100-fold or more over than the normal control level. Alternatively, the level of analyte is decreased 10%, 15%, 25%, 50%, 75%, 90%, 95%, 99%, 99.9% or 99.99% or more fold compared to the control level.

The patient derived sample is any sample from a test subject, e.g., a patient known to or suspected of having a cardiovascular disorder. For example, the sample is blood, or cardiovascular tissue. Blood includes serum, plasma, or other blood products or components. The blood is obtained from the portal vein. Alternatively, the blood is obtained from the peripheral circulation. Cardiovascular tissue includes tissue isolated from the heart and associated blood vessels (e.g., the aorta and the portal vein)

The invention further provides methods of assessing the efficacy of a treatment of a cardiovascular disorder in a subject, by determining a level of a CVD-associated analyte in a patient derived sample, and comparing the level to a normal control level. An increase in CVD26-39, CVD55-74, and/or CVD99-112 in the patient derived sample compared to a normal control level indicates that the treatment is efficacious. Alternatively, a decrease in CVD1-25, CVD40-54, and/or CVD75-98 in the patient derived sample compared to a normal control level indicates that the treatment is efficacious.

In another aspect the invention provides methods of identifying an agent that modulates the onset or progression of a cardiovascular disorder in a subject. The method includes contacting the subject with a candidate agent, and determining a test level of an analyte in a sample derived from the subject. The test level is compared with a reference level of the analyte. An alteration, e.g., an increase or decrease of the test level relative to the reference level, indicates that the test agent modulates the onset or progression of a cardiovascular disorder. The reference level is derived from a sample from the subject. Alternatively, the reference level is derived from a database.

Also included in the invention is a kit having a detection reagent that identifies two or more of CVD 1-112.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms of a cardiovascular disease, e.g., a myocardial infarction. Other features and advantages of the invention will be apparent from the following detailed description, and from the claims.

### DETAILED DESCRIPTION

The present invention is based, in part, on the discovery of changes of analyte levels in blood samples from patients undergoing alcohol septal ablation for hypertrophic cardiomyopathy, which results in an induced myocardial infarction (MI). The differences in analyte levels are identified by analyzing the relative concentrations of large sets of small molecules using mass spectrometry to create biochemical profiles for individual samples. Such profiles are then compared to identify biochemical changes that occur in MI patients over a period of twenty-four hours, as compared to the individual patients prior to onset of MI. Statistical and bioinformatic analyses of these profiles identifies patterns of change for the small molecules measured. These patterns of change form the basis for biochemical signatures that are characteristic for cardiovascular disorders. These signatures can then be used to predict the presence and progression of cardiovascular disorders, as well as the toxicological and clinical behavior of new drug candidates to treat or prevent cardiovascular disorders.

These sets of small molecules measured herein are the end result of all the regulatory complexity present in the cell or tissue (transcriptional regulation, translational regulation, post-translational modification, cellular localization of the proteins, and partitioning of substrates and cofactors relative to the proteins), and they summarize all of those different levels of regulation. By monitoring hundreds of blood or tissue constituents rather than the tens measured in routine clinical diagnostic tests, the existence of, or predisposition to, cardiovascular disorders can be diagnosed.

The analyte profiles of samples from 17 subjects with induced myocardial infarctions were analyzed as described in Examples 1 and 2. By comparing analyte patterns in patients experiencing myocardial infarctions with the same patients prior to onset of myocardial infarction, 64 analytes were identified as being commonly increased in myocardial infarction and 48 analytes were identified as being commonly decreased in myocardial infarction.

The present invention also provides for a comparison of analyte patterns in patients suffering from unstable angina with the same patients following treatment with a putative therapeutic agent.

The present invention also provides for analysis of analyte patterns in patients undergoing exercise testing for ischemic heart disease.

The differentially modulated analytes identified herein are used for diagnostic purposes as markers of cardiovascular disorders.

The analytes whose levels are modulated (i.e., increased or decreased) in patients experiencing myocardial infarction between the time of infarct and one hour are summarized in Table 1. The analytes whose levels are modulated in patients experiencing myocardial infarction between the time of infarct and four hours are summarized in Table 2. The analytes whose levels are modulated in patients experiencing myocardial infarction between the time of infarct and twenty-four hours are summarized in Table 3. These analytes are collectively referred to herein as “CVD-associated analytes.” Unless indicated otherwise, “CVD” is meant to refer to any of the analytes disclosed herein (e.g., CVD 1-112). The analytes that have been previously described are identified by chemical name. For those analytes that have not heretofore been described are identified by parent, daughter mass and collision energy. Exemplary separation conditions are described in the Examples below. With this information those skilled in the art can readily identify a CVD-associated analyte in a sample. For example, parent and daughter masses, and collision energies are used to set up a mass spectrometer. The column type and the mobile phase conditions described in the Examples are used to set up the HPLC step. For the majority of MI-associated analytes, a single peak that changes with MI will be visible in the chromatogram from human blood. Where more than one peak is visible, the desired peak is the one that shows a change between diseased and normal samples.

TABLE 1
Relative analyte composition in one-hour post-myocardial
infarction vs. pre-myocardial infarction levels

| | | One-hour post MI | | | Collision | |
|---|---|---|---|---|---|---|
| | | level relative to pre- | Analyte | Fragment | Energy (in | |
| CVD# | Chemical Name | MI level | Mass | Mass | Volts) | Column |
| 1 | CAP27 | greater | 201 | 121.2 | −30 | 4 |
| 2 | CAP28 | greater | 227.3 | 127 | −24 | 3 |
| 3 | CAP274 | greater | 173 | 129.2 | −12 | 4 |
| 4 | CAP293 | greater | 231.2 | 151.4 | −24 | 4 |
| 5 | CAP30 | greater | 233.1 | 151 | −17 | 3 |
| 6 | CAP24 | greater | 133 | 79.2 | −32 | 3 |
| 7 | Hippuric Acid | greater | 177.9 | 133.9 | −16 | 3 |
| 8 | CAP37 | greater | 305.2 | 141 | −17 | 3 |
| 9 | CAP269 | greater | 178 | 133.9 | −17 | 4 |
| 10 | Lysine/Glutamine | greater | 146.9 | 130 | 16 | 1 |
| 11 | CAP322 | greater | 179 | 135 | −16 | 4 |
| 12 | CAP308 | greater | 179 | 74.9 | −24 | 4 |
| 13 | CAP332 | greater | 176.9 | 87 | −19 | 4 |
| 14 | Citric Acid | greater | 191 | 87 | −25 | 3 |
| 15 | CAP289 | greater | 194.9 | 75 | −29 | 4 |
| 16 | Ribose-5-P/Ribulose-5-P | greater | 229.3 | 96.9 | −20 | 4 |
| 17 | Isocitric Acid | greater | 191 | 172.9 | −18 | 3 |
| 18 | Taurocholic Acid | greater | 514.4 | 123.8 | −75 | 3 |
| 19 | CAP31 | greater | 235.1 | 151.2 | −20 | 3 |
| 20 | CAP20 | greater | 520.3 | 184.2 | 33 | 1 |
| 21 | Gluconorate | greater | 192.9 | 102.9 | −15 | 3 |
| 22 | CAP40 | greater | 387.5 | 141.2 | −24 | 3 |
| 23 | Homogentisic Acid | greater | 166.9 | 79 | −15 | 3 |
| 24 | CAP47 | greater | 308.7 | 145 | −28 | 4 |
| 25 | Oxaloacetate | greater | 130.9 | 87 | −20 | 3 |
| 26 | Tryptophan | less | 204.8 | 187.9 | 16 | 1 |
| 27 | Phenylalanine | less | 166 | 119.9 | 17 | 1 |
| 28 | Xanthosine | less | 285.2 | 153.2 | 13 | 2 |
| 29 | CAP3 | less | 228.1 | 111.8 | 12 | 1 |
| 30 | CAP294 | less | 189.8 | 110.3 | −24 | 4 |
| 31 | 2′-deoxyadenosine | less | 252 | 136.2 | 28 | 1 |
| 32 | Kyurenic Acid | less | 207 | 144 | −33 | 3 |
| 33 | CAP266 | less | 188.8 | 109.3 | −33 | 4 |
| 34 | CAP11 | less | 317.9 | 105.1 | 12 | 1 |
| 35 | CAP19 | less | 515.1 | 269.1 | 25 | 1 |
| 36 | Aspartate | less | 134 | 74.1 | 21 | 1 |
| 37 | Glycochenodeoxycholic | less | 448.4 | 74 | −50 | 3 |
| | Acid | | | | | |
| 38 | CAP307 | less | 245 | 164.9 | −30 | 4 |
| 39 | Betaine | less | 117.9 | 58 | 29 | 1 |

TABLE 2
Relative analyte composition in four-hour post-myocardial
infarction vs. pre-myocardial infarction levels

| | | Four-hour post MI | | | Collision | |
|---|---|---|---|---|---|---|
| | | level relative to pre- | Analyte | Fragment | Energy (in | |
| CVD# | Chemical Name | MI level | Mass | Mass | Volts) | Column |
| 40 | Taurocholic Acid | greater | 514.4 | 123.8 | −75 | 3 |
| 41 | CAP274 | greater | 173 | 129.2 | −12 | 4 |
| 42 | CAP24 | greater | 133 | 79.2 | −32 | 3 |
| 43 | CAP33 | greater | 238.8 | 89.1 | −16 | 3 |
| 44 | CAP293 | greater | 231.2 | 151.4 | −24 | 4 |
| 45 | CAP38 | greater | 321.4 | 140.8 | −14 | 3 |
| 46 | CAP37 | greater | 305.2 | 141 | −17 | 2 |
| 47 | Homogentisic Acid | greater | 166.9 | 79 | −15 | 3 |
| 48 | CAP40 | greater | 387.5 | 141.2 | −24 | 3 |
| 49 | CAP28 | greater | 227.3 | 127 | −24 | 3 |
| 50 | Gluconorate | greater | 192.9 | 102.9 | −15 | 3 |
| 51 | Ribose-5-P/Ribulose-5-P | greater | 229.3 | 96.9 | −20 | 4 |
| 52 | CAP23 | greater | 130 | 87.7 | −15 | 3 |
| 53 | Cytosine/Histamine | greater | 111.9 | 95 | 20 | 2 |
| 54 | CAP41 | greater | 170.7 | 89.1 | −15 | 4 |
| 55 | CAP266 | less | 188.8 | 109.3 | −33 | 4 |
| 56 | CAP294 | less | 189.8 | 110.3 | −24 | 4 |
| 57 | Valine | less | 117.9 | 72 | 27 | 1 |
| 58 | CAP307 | less | 245 | 164.9 | −30 | 4 |
| 59 | CAP2 | less | 204.3 | 84.9 | 25 | 1 |
| 60 | Proline | less | 115.9 | 69.9 | 18 | 1 |
| 61 | CAP269 | less | 178 | 133.9 | −17 | 4 |
| 62 | Homoserine | less | 120.1 | 56.2 | 28 | 2 |
| 63 | Carnitine | less | 162.8 | 103 | 27 | 2 |
| 64 | Trimethylamine-N-oxide | less | 76 | 58 | 29 | 2 |
| 65 | Tryptophan | less | 204.8 | 187.9 | 16 | 1 |
| 66 | Threonine | less | 120 | 74 | 18 | 2 |
| 67 | Hippuric Acid | less | 177.9 | 133.9 | −16 | 3 |
| 68 | Methionine | less | 149.9 | 103.9 | 17 | 2 |
| 69 | Hydroxyproline | less | 132 | 68 | 20 | 2 |
| 70 | Homocysteine | less | 135.8 | 90 | 20 | 2 |
| 71 | Allantoin | less | 159 | 116 | 10 | 1 |
| 72 | CAP14 | less | 344.8 | 105.1 | 29 | 1 |
| 73 | Guanidinoacetic Acid | less | 118 | 101 | 15 | 1 |
| 74 | CAP8 | less | 262.8 | 104.8 | 17 | 1 |

TABLE 3
Relative analyte composition in twenty-four hour post-myocardial
infarction vs. pre-myocardial infarction levels

| | | Twenty-four hour | | | Collision | |
|---|---|---|---|---|---|---|
| | | post MI level relative | Analyte | Fragment | Energy (in | |
| CVD# | Chemical Name | to pre-MI level | Mass | Mass | Volts) | Column |
| 75 | CAP286 | greater | 217 | 136.8 | −21 | 4 |
| 76 | CAP293 | greater | 231.2 | 151.4 | −24 | 4 |
| 77 | CAP319 | greater | 194.9 | 98.9 | −22 | 4 |
| 78 | CAP21 | greater | 104.1 | 74.1 | −16 | 3 |
| 79 | Taurocholic Acid | greater | 514.4 | 123.8 | −75 | 3 |
| 80 | CAP38 | greater | 321.4 | 140.8 | −14 | 3 |
| 81 | CAP24 | greater | 133 | 79.2 | −32 | 3 |
| 82 | CAP23 | greater | 130 | 87.7 | −15 | 3 |
| 83 | Alpha-Keto-Glutarate | greater | 144.8 | 101 | −15 | 3 |
| 84 | Glycoholic Acid | greater | 464.4 | 74 | −50 | 3 |
| 85 | CAP33 | greater | 238.8 | 89.1 | −16 | 3 |
| 86 | CAP30 | greater | 233.1 | 151 | −17 | 3 |
| 87 | CAP37 | greater | 305.2 | 141 | −17 | 3 |
| 88 | CAP31 | greater | 235.1 | 151.2 | −20 | 3 |
| 89 | CAP40 | greater | 387.5 | 141.2 | −24 | 3 |
| 90 | Glycochenodeoxycholic | greater | 448.4 | 74 | −50 | 3 |
| | Acid | | | | | |
| 91 | CAP22 | greater | 123.9 | 79.9 | −30 | 3 |
| 92 | Creatine | greater | 131.9 | 90 | 15 | 2 |
| 93 | Serine | greater | 105.9 | 60.1 | 15 | 2 |
| 94 | Tyrosine | greater | 181.8 | 135.9 | 17 | 2 |
| 95 | Dimethyl Glycine | greater | 103.9 | 58.1 | 10 | 2 |
| 96 | Asparagine | greater | 132.9 | 74 | 22 | 1 |
| 97 | Phenylalanine | greater | 166 | 119.9 | 17 | 1 |
| 98 | Methionine | greater | 149.9 | 103.9 | 17 | 1 |
| 99 | CAP316 | less | 153.1 | 109 | −21 | 4 |
| 100 | CAP269 | less | 178 | 133.9 | −17 | 4 |
| 101 | CAP287 | less | 225 | 127 | −26 | 4 |
| 102 | CAP336 | less | 198.8 | 119.3 | −27 | 4 |
| 103 | CAP268 | less | 173 | 92.7 | −34 | 4 |
| 104 | CAP284 | less | 173.1 | 80 | −34 | 4 |
| 105 | Hydroxyproline | less | 132 | 68 | 20 | 2 |
| 106 | N-Carbamyl-Beta- | less | 132.9 | 90 | 17 | 2 |
| | Alanine | | | | | |
| 107 | CAP20 | less | 520.3 | 184.2 | 33 | 1 |
| 108 | CAP6 | less | 258.2 | 104.1 | 20 | 1 |
| 109 | CAP11 | less | 317.9 | 105.1 | 12 | 1 |
| 110 | CAP3 | less | 228.1 | 111.8 | 12 | 1 |
| 111 | CAP2 | less | 204.3 | 84.9 | 25 | 1 |
| 112 | CAP7 | less | 261 | 96.7 | 14 | 1 |

Analytes having chemical names “CAP” (e.g., CAP7) are novel compounds without chemical names. These compounds can be identified by one or ordinary skill in the art using the provided analyte mass, the fragments mass, and the collision energy.

The term “analyte” includes organic and inorganic molecules that are present in the tissue, fluid, cell, cellular compartment, or organelle. An analyte includes signaling molecules and intermediates in the chemical reactions that transform energy derived from food into usable forms. The term “metabolite” includes any chemical or biochemical product of a metabolic process, such as any compound produced by the processing, cleavage or consumption of a biological molecule (e.g., a protein, carbohydrate, or lipid). The term “metabolome” includes all of the analytes present in a given organism. The metabolome includes both metabolites as well as products of catabolism.

By measuring the level of the various analytes in a sample of cells, tissue or biological fluid, cardiovascular disorders are diagnosed. Similarly, measuring the level of these analytes in response to various agents can identify agents for treating cardiovascular disorders.

The invention involves determining (e.g., measuring) the level of at least one, and up to all the analytes listed in Tables 1-3. Optionally, the CVD-associated analyte is determined in a sample by detecting one or more metabolites of the analyte in the sample. Using molecular mass information and collision energy provided herein, the CVD associated-analytes are detected and measured using techniques well known to one of ordinary skill in the art. For example, CVDs 1-112 are detected by mass spectrometric analysis.

The level of one or more of the CVD-associated analytes in the test sample, e.g., a patient derived sample, is then compared to levels of the same analytes in a reference population. The reference population includes one or more reference samples. A reference sampe is a sample for which the compared parameter is known, i.e., cardiovascular disorder sample or normal (non-cardiovascular disorder sample).

Whether or not a pattern of analyte levels in the test sample (e.g. patient derived sample) compared to the reference population indicates a cardiovascular disorder or predisposition thereto depends upon the composition of the reference population. For example, if the reference population is composed of non-CVD samples, a similar analyte pattern in the test sample and reference population indicates the test sample is non-CVD. Conversely, if the reference population is made up of a CVD sample, a similar analyte pattern between the test sample and the reference population indicates that the test sample includes CVD.

A level of expression of a CVD analyte in a test sample is considered altered in levels if the level of the analyte is increased 1.1-fold, 1.25-fold, 1.5-fold, 1.75-fold, 2-fold, 5-fold, 10-fold, 25-fold, 1 00-fold or more over the reference population Alternatively, the level of analyte is decreased 10%, 15%, 25%, 50%, 75%, 90%, 95%, 99%, 99.9% or 99.99% or more fold compared to the reference population.

Differential analyte levels between a test sample and a reference population is normalized to a control analyte. For example, a control analyte is one that is known not to differ depending on disease state of the population.

The test sample is compared to multiple reference populations. Each of the multiple reference populations may differ in the known parameter. Thus, a test sample may be compared to a second reference population known to contain, e.g., a patient suffering from MI, as well as a second reference population known to contain, e.g., individuals not suffering from MI.

The test sample is obtained from a bodily tissue (e.g., cardiovascular tissue) or a bodily fluid, e.g., a biological fluid (such as blood). For example, the test sample is obtained from blood drawn from the portal vein. Alternatively, the sample is obtained from cardiac tissue, such as the entire tissue, entire cell or from specific cellular compartments such as the cytoplasm, the mitochondria, the Golgi apparatus, the endoplasmic reticulum, the nucleus, or the cytosol. The sample is substantially free of macromolecules (e.g., large proteins and polynucleotides with molecular weights of greater than 10,000).

The reference population is the individual prior to onset of a cardiovascular disorder, such as myocardial infarction. Alternatively, the reference population is derived from a tissue or fluid type similar to test sample. Optionally, the control population is derived from a database of molecular information derived from samples for which the assayed parameter or condition is known.

The subject is preferably a mammal. The mammal can be, e.g., a human, non-human primate, mouse, rat, dog, cat, horse, or cow. Small and large animal models of cardiovascular disease are included in the present invention.

Analytes disclosed herein are detected in a variety of ways known to one of skill in the art, including the refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, MALDI combined with mass spectroscopy, ion spray spectroscopy combined with mass spectroscopy, capillary electrophoresis, NMR and IR detection.

Diagnosing Cardiovascular Disorders

A cardiovascular disorder is diagnosed by measuring the level of one or more CVD-associated analytes from a test sample (i.e., a patient derived sample such as blood or cardiovascular tissue). The level or expression of one or more CVD-associated analytes, e.g., CVD 1-112 is determined in the test sample and compared to the level or expression of the normal control level. A normal control level is a profile of CVD-associated analytes typically found in a population known not to be suffering from CVD. This population includes individuals prior to induction of a myocardial infarction, as described in Example 1. An increase or a decrease of the level of expression in the patient derived tissue sample of the CVD-associated analytes indicates that the subject is suffering from or is at risk of developing CVD. For example, an increase in expression of CVD 1-25, CVD 40-54, or CVD 75-98 in the test sample compared to the normal control level indicates that the subject is suffering from or is at risk of developing CVD. Conversely, a decrease in expression of CVD 26-39, CVD 55-74, or CVD 99-112 in the test sample compared to the normal control level indicates that the subject is suffering from or is at risk of developing CVD.

When one or more of the CVD-associated analytes are altered in the test sample compared to the normal control level indicates that the subject suffers from or is at risk of developing CVD. For example, at least 1%, 5%, 25%, 50%, 60%, 80%, 90% or more of the panel of CVD-associated analytes (CVD1-1 12), are altered.

Assessing Efficacy of Treatment of CVD in a Subject

The CVD-associated analytes identified herein also allow for the course of treatment of CVD to be monitored. In this method, a test sample is provided from a subject undergoing treatment for CVD. If desired, test cell populations are obtained from the subject at various time points before, during, or after treatment. Expression of one or more of the CVD-associated analytes, in the cell population or biological fluid is then determined and compared to a reference cell population or biological fluid which includes cells or biological fluid whose CVD state is known. The reference cells or fluid have not been exposed to the treatment.

If the reference population contains a non-CVD sample, a similarity in expression between CVD-associated analyte in the test sample and the reference population indicates that the treatment is efficacious. However, a difference in expression between a CVD-associated analyte in the test sample and a normal control reference population indicates a less favorable clinical outcome or prognosis.

By “efficacious” is meant that the treatment leads to a reduction in a pathologically increased analyte or an increase of a pathologically decreased analyte or a decrease in size, or prevalence, of myocardial infarction in a subject. When treatment is applied prophylactically, “efficacious” means that the treatment retards or prevents a CVD from forming or retards, prevents, or alleviates a symptom of clinical CVD. Assessment of CVD is made using standard clinical protocols. Efficaciousness is determined in association with any known method for diagnosing or treating CVD. CVD is diagnosed for example, by identifying symptomatic anomalies, e.g., chest pain at rest that spreads to one or both arms, the back or the neck, that may be accompanied by dizziness, fainting, nausea or shortness of breath.

Assessing the Prognosis of a Subject with Myocardial Infarction

Also provided is a method of assessing the prognosis of a subject with CVD by comparing the level of one or more CVD-associated analyte in a test sample to the level of the analytes in a reference population derived from patients over a spectrum of disease stages. By comparing analyte level of one or more CVD-associated analyte in the test sample and the reference population(s), or by comparing the pattern of analyte levels over time in test samples derived from the subject, the prognosis of the subject can be assessed.

A decrease in expression of one or more of CVD 26-39, CVD 55-74, or CVD 99-112 compared to a normal control or an increase of expression of one or more of CVD 1-25, CVD 40-54, or CVD 75-98 compared to a normal control indicates less favorable prognosis. An increase in expression of one or more of CVD 26-39, CVD 55-74, or CVD 99-112 indicates a more favorable prognosis, and a decrease in expression of one or more of CVD 1-25, CVD 40-54, or CVD 75-98 indicates a more favorable prognosis for the subject.

Kits

The invention also includes a CVD associated analyte-detection reagent in the form of a kit. For example, the kit includes a labeled compound or agent that of detects the CVD-associated analyte in a biological sample. The kit further includes a means for determining the amount of the analyte in the sample (e.g., an antibody, molecular or chemical sensor against the CVD associated analyte). Optionally, the kit contains, e.g., a buffering agent, a preservative, a stabilizing agent, or components necessary for detecting the detectable agent (e.g., a substrate). The kit contains a control sample or a series of control samples that can be assayed and compared to the test sample contained. Each component of the kit is enclosed within an individual container and all of the various containers are within a single package along with instructions for observing whether the tested subject is suffering from or is at risk of developing a disorder associated with the CVD-associated analyte. For example, the kit comprises two or more CVD1-1 12 along with detection means and instructions for use thereof.

This invention is further illustrated by the following examples that should not be construed as limiting. The contents of all references and published patents and patent applications cited throughout the application are hereby incorporated by reference.

### EXAMPLE 1

### Isolation of Biological Materials From Patients Having Induced Myocardial Infarctions

Patients undergoing alcohol septal ablation for hypertrophic cardiomyopathy results in an induced myocardial infarction. Patients suffering from hypertrophic cardiomyopathy are subjected to a procedure in which a catheter is placed into one of the vessels supplying the cardiac septum. A balloon is inflated to damage the vessel wall and a small amount of alcohol is infused at the site to induce clot formation. The resulting infarction causes the death of a portion of the hypertrophic tissue and provides relief for the cardiomyopathy. Whole blood, plasma, serum, or other blood components or fractions are useful in the methods of the present invention.

Serum samples are drawn from each patient at the start of the procedure (“time zero”), one-hour, four hours, and twenty-four hours after the infarction. For some patients, samples are also drawn from the portal vein through a catheter during the procedure on the theory that direct products from the heart are present in blood from the portal vein at higher concentrations than in peripheral blood. It is an advantage of the present invention to separate the direct effects from the systemic response to the infarct.

The samples thus obtained were used to identify a biochemical signature that might be associated with cardiac ischemia or myocardial infarction by analyzing the serum for some 400 biochemicals, as described in Example 2. Analysis of the data using penalized discriminant analysis reveals that there are substantial differences between the time points, with the largest difference between time zero and 1 hour post-infarct, as shown in Tables 1-3.

During the alcohol septal ablation treatment, levels of analytes in the patient's blood or cardiovascular tissue following myocardial infarction are compared with analyte levels in blood or tissue prior to onset of the myocardial infarction. Therefore, the patient serves as his/her own control. It is useful to compare blood samples obtained from alcohol septal ablation treatment with samples taken during balloon angiography procedures or other cardiac catheterizations in humans. This comparison allows the separation of the MI-specific changes from procedure-associated changes.

Non-induced myocardial infarctions in humans are the result of acute and/or chronic events. Therefore, induction of a myocardial infarction in an otherwise healthy heart may not identify all CVD-associated analytes. Samples are also obtained from patients with suspected myocardial infarction for testing of the applicability of the CVD-associated analyte signature.

The alcohol septal ablation treatment procedure will in some patients be based on the extent of the cardiac hypertrophy, the gender, age and health of the patient. Surgeon- and/or facility-specific variations in the procedure may also impact the CVD-associated analyte signature obtained following induced MI. Therefore, samples can be obtained from procedures performed by multiple surgeons operating at multiple facilities.

### EXAMPLE 2

### Detecting CVD-associated Analytes

CVD-associated analytes or metabolites are detected using a single technique or a combination of techniques for separating and/or identifying analytes known in the art. Examples of separation and analytical techniques which are used to separate and identify the CVD-associated analyte in a sample include mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), and Light Scattering analysis (LS). The methods of the invention detect both electrically neutral as well as electrochemically active compounds. Preferably, the separation and detection of CVD associated analytes is accomplished by MS. Detection and analytical techniques can be arranged in parallel to optimize the number of molecules identified.

For example, mass spectroscopy is used as a method for detecting and quantifying the analytes contained in a biological source (e.g., blood) taken from a subject. The analytes from a subject are separated through the use of column chromatography. Multiple columns are used as shown in Table 4, with each column designed to separate classes of compounds. In this format, there can be a column switching valve which allows for staggered injections into the multiple columns. This format is described below and in U.S. patent application Ser. No. 10/323,493, the contents of which are incorporated by reference herein.

TABLE 4
Column chromatographic separation of analytes

| Column # | Column type | Aqueous Mobile Phase | Organic Mobile Phase |
|---|---|---|---|
| 1 | AA: Phenomenex Luna | 0.1% acetic acid in | 0.1% acetic acid in |
| | Phenyl-Hexyl 4.60 mm | water | acetonitrile |
| | diameter × 75 mm length | | |
| 2 | NA: Phenomenex Luna | 0.1% acetic acid in | 0.1% acetic acid in |
| | Phenyl-Hexyl 2.00 mm | water | acetonitrile |
| | diameter × 100 mm length | | |
| 3 | NO: Phenomenex Synergi | 5 mM ammonium | 5 mM ammonium |
| | Polar RP 2.00 mm | acetate in wate | acetate in acetonitrile |
| | diameter × 150 mm length | | |
| 4 | SN: Phenomenex Luna | 10 mM ammonium | 10 mM ammonium |
| | Amino (NH2) | acetate, 0.25% | acetate, 0.25% |
| | 4.60 mm diameter × 50 mm | triethylamine in | triethylamine |
| | length | water | |

Sample Collection:

Serum: Serum obtained from whole blood using standard techniques is extracted in order to inactivate bloodborne pathogens. An examplary extraction protocol is as follows: 1/20 volume of formic acid is added to the serum, incubated at room temperature for 30 minutes. Two volumes of acetonitrile is then added, mixed well and then subjected to centrifugation. The supernatant is recovered and dried by evaporation under nitrogen gas. The resulting residue is resuspended in Internal Standard Solution as described below.

Plasma: Whole blood is collected into an anti-coagulant (heparin or citrate/EDTA). Cells are removed by centrifugation. The plasma layer above the cells is removed to a new tube containing chelating agent and antioxidant are added (EDTA, final concentration 0.4mM; TEMPO, final concentration 0.8 mM). The sample is extracted immediately, or stored at −80° C. until extraction.

Sample Preparation:

The biological fluids or cells (e.g. blood, cardiovascular tissue, a suspension of cells, etc.) arrayed in a 96-well plate are mixed with an equal volume of extraction solvent (e.g. 90/10 Acetonitrile/water, 1% trifluoroacetic acid) and vortexed for 60 seconds. If using soft-tissues (e.g. heart tissue, isolated blood vessels, biopsy material), the tissue is homogenized at 4° C. using a Teflon-on-glass or other appropriate homogenizer in an equal volume of extraction solvent. The resulting solution or homogenate from the above steps is centrifuged at 3,000 g for 15 minutes to remove precipitated proteins and other macromolecules. 100 μl of the supernatant is transferred to a new 96-well plate and dried under Nitrogen. The dried sample is then stored at −80° C., until ready for analysis, at which time it is reconstituted with the Internal Standard solution (Stable isotopic and/or deuteriated compounds e.g., Glucose-d7, Valine-d8, glycerol-d8 in 50/50 acetonitrile/water). Alternatively, a biological fluid can be used directly, after dilution with the Internal Standard solution.

The platform detects the presence of molecules from a defined list of biochemical compounds (See, e.g., Tables 1-3) and only from this list. Other molecules present in the sample are not detected. This platform is used to create signatures whose components are biochemical compounds that can, in combination, distinguish between classes of samples. Because the identities of the compounds are known, the composition of signatures can be subject to biological interpretation.

There are seven components to the platform: 1—8 HPLC pumps used to deliver liquid phases; 2—A 4-injector autosampler for controlling sample injection; 3—up to four different HPLC columns for separation; 4—A switching valve used to control column to MS transfer; 5—An LC/MS interface such as electrospray (ES), atmosphere pressure chemical ionization (APCI) for connection of HPLC and MS; 6—A triple quadrupole mass spectrometer for compound separation and identification; 7—A computer for instrument control and data acquisition.

The columns are indicated in Table 4.

The column switching valve allows staggered injection into the multiple columns, and the effluent from the different columns to be analyzed sequentially in a single run. This way data from 4 columns can be captured from single sample on a single mass spectrometer, rather than needing 4 separate runs. Compounds with distinct masses but similar retention times can be separated by the mass spectrometer. The targeted compounds (Tables 1-3) are each detected by the MS throughout the run to produce a series of mass chromatograms.

Mass Chromatogram Processing

Biochemical Compound Identification: In order to quantify a single desired biochemical compound, the triple quadrupole mass spectrometer combines two mass filters and a fragmentation step. The first quadrupole acts as a mass filter and only allows ions of a particular mass/charge ratio to proceed fuirther into the second quadrupole. This second chamber acts as the collision cell where the filtered molecules are fragmented with gas molecules and with a source of electrons. This fragmentation causes each parent biochemical molecule to fragment in a predictable manner producing fragment (or daughter) ions of a particular mass. The third quadrupole acts as a second mass filter and only allows the desired daughter ions to pass through to the detector. Thus the combination of the two mass filters allow for quantitation of only molecules with the desired mass/charge ratio that produce daughter ions of the desired mass. In most cases this will detect only a single compound. Distinct biochemical compounds that have identical parent and daughter masses will be ambiguous, and for those situations, it may be possible to use the initial step of liquid chromatography to separate the molecules by retention time.

In order to detect and quantify over 400 target compounds from a single sample, the parent and daughter ion masses of each compound are programmed into the machine. The two mass filters rapidly cycle through these mass combinations, detecting each of the target compounds as the sample comes off the columns.

Biochemical Compound Quantitation: After peak identification, the amount of each compound must be calculated. This is achieved by the step of peak integration. The area under the peak for each of the target compounds is calculated using the AB Analyst software. These values are then scaled by the area of the internal standard peak, producing a relative peak area ratio.

QC: In addition to standard processing, each sample is run through a suite of QC procedures which examine (among other things) retention times, and peak areas for internal standards for indications of problems with the LC/MS process. In addition, individual peaks can be flagged for manual examination if parameters (such as for peak shape) exceed normal bounds.

### EXAMPLE 3

### Isolation of Biological Materials from Mammalian Models of Myocardial Infarction

The present invention also provides non-human mammalian models of cardiovascular disorders, including myocardial infarction and stroke. Since the specific events occuring during a given cardiovascular disorder are unique to each human patient, the patient serves as his/her own control. Thus, comparisons in analyte levels must be made back to a time before the onset of the cardiovascular event (zero time). The use of multiple non-human animals such as rodents allows for the control and exclusion of analytes not associated with cardiovascular disorders, such as those whose levels are modulated by diurnal effects, effects of anesthesia and/or other medications used during the procedure, food effects, or other variable influencing factors that may impact the patient over time. When alcohol septal ablation for hypertrophic cardiomyopathy is performed on non-human mammals, a sham-operated animal where saline is infused rather than alcohol, such that no clot is initiated, is used to control for the effects described above.

### EXAMPLE 4

### Complex Biomolecules and Non-polar Analytes

The present invention includes methods that examine 400 or more biochemicals in order to obtain a biochemical signature predictive of cardiovascular disorders. Complex molecules, such as proteins (e.g., enzymes), nucleic acids, and carbohydrates are also examined by these methods. Additionally, non-polar compounds whose levels are modulated during myocardial infarction or ischemia are amenable to study. These complex molecules and non-polar compounds may be novel biochemicals, or may be known from the literature. Identification of one or more members of a cellular pathway (e.g., a biochemical pathway) suggests that other members of the same pathway will be useful to validate the quality and specificity of the signature. By way of non-limiting example; useful proteins include creatine kinase; single and serial measurements; creatine kinase subunit; single and serial measurements; Troponin T; Troponin I; P-selectin; fatty acid binding protein; myoglobin carbonic anhydrase III; vitamin E; S100B; N-terminal pro-brain natriuretic peptide; myosin light chain-1; mineralocorticoid receptor; endothelin-1 receptor; C-reactive protein; lipoprotein (a); homocysteine; retinol; alpha- and gamma-tocopherols; creatine kinase-M.

Other suitable biochemicals are described in the following references, which are incorporated herein in their entireties.

Svensson, L., C. Axelsson, et al. (2003). “Elevation of biochemical markers for myocardial damage prior to hospital admission in patients with acute chest pain or other symptoms raising suspicion of acute coronary syndrome.” J Intern Med 253(3): 311-9.

Svensson, L., L. Isaksson, et al. (2003). “Predictors of myocardial damage prior to hospital admission among patients with acute chest pain or other symptoms raising a suspicion of acute coronary syndrome.” Coron Artery Dis 14(3): 225-31.

Kennon, S., C. P. Price, et al. (2003). “Cumulative risk assessment in unstable angina: clinical, electrocardiographic, autonomic, and biochemical markers.” Heart 89(1): 36-41.

Iliou, M. C., C. Fumeron, et al. (2003). “Prognostic value of cardiac markers in ESRD: Chronic Hemodialysis and New Cardiac Markers Evaluation (CHANCE) study.” Am J Kidney Dis 42(3): 513-23.

Hubbard, J. (2003). “Common biochemical markers for diagnosing heart disease.” Nurs Times 99(26): 24-5.

Ottani, F., M. Galvani, et al. (2002). “[Markers of myocardial damage in the diagnosis of acute myocardial infarction: the Italian reality in the year 2000].” Ital Heart J 3(9 Suppl): 933-42.

Holmvang, L., B. Jurlander, et al. (2002). “Use of biochemical markers of infarction for diagnosing perioperative myocardial infarction and early graft occlusion after coronary artery bypass surgery.” Chest 121(1): 103-11.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A cardiovascular disorder reference profile, comprising a pattern of two or more analytes or metabolites thereof, selected from the group consisting of CVD 1-112.

2. A cardiovascular disorder reference profile, comprising a pattern of two or more analytes or metabolites thereof, selected from the group consisting of CVD 1-25, 40-54 and 75-98.

3. A cardiovascular disorder reference profile, comprising a pattern of two or more analytes or metabolites thereof, selected from the group consisting of CVD 26-39, 55-74, and 99-112.

4. A method of diagnosing a cardiovascular disorder (CVD) or a predisposition to developing cardiovascular disorder in a subject, comprising determining a level of a CVD-associated analyte in a patient derived sample, wherein an increase or decrease of said level compared to a normal control level indicates that said subject suffers from or is at risk of developing a cardiovascular disorder.

5. The method of claim 4, wherein said CVD-associated analyte is selected from the group consisting of CVD 1-25, 40-54 and 75-98, wherein an increase in said level compared to a normal control level indicates that said subject suffers from or is at risk of developing myocardial infarction.

6. The method of claim 4, wherein said CVD-associated analyte is selected from the group consisting of CVD 1-25, 40-54 and 75-98, wherein an increase in said level compared to a normal control level indicates that said subject suffers from or is at risk of having a stroke.

7. The method of claim 4, wherein said analyte is selected from the group consisting of CVD 26-39, 55-74, and 99-112 wherein a decrease in said level compared to a normal control level indicates that said subject suffers from or is at risk of developing myocardial infarction.

8. The method of claim 5, wherein said increase is at least 1.1-fold greater than said normal control level.

9. The method of claim 7, wherein said decrease is at least 10% less than said normal control level.

10. The method of claim 4, wherein said sample is blood or cardiovascular tissue.

11. The method of claim 4, wherein said cardiovascular disorder is high blood pressure, coronary heart disease, stroke, congestive heart failure, unstable angina or hypertrophic cardiomyopathy.

12. The method of claim 4, wherein said method further comprises determining said level of a plurality of CVD-associated analytes.

13. A method of assessing the efficacy of a treatment of a cardiovascular disorder in a subject, comprising determining a level of a CVD-associated analyte in a patient derived sample, and comparing said level to a normal control level thereby monitoring the treatment of the cardiovascular disorder in said subject.

14. The method of claim 13, wherein said CVD-associated analyte is selected from the group consisting of CVD 26-39, 55-74, and 99-112, wherein an increase in said level compared to a said normal control level indicates that treatment is efficacious

15. The method of claim 13, wherein said CVD-associated analyte is selected from the group consisting of CVD 1-25, 40-54 and 75-98, wherein decrease in said level compared to a said normal control level indicates that treatment is efficacious

16. A method of identifying an agent that modulates the onset or progression of a cardiovascular disorder in a mammalian subject, comprising:
i) contacting said subject with a candidate agent;
ii) determining a test level of an analyte in a sample derived from said subject following said contacting;
iii) comparing said test level with a reference level of said analyte, wherein an increase or decrease of said test level relative to said reference level indicates that said test agent modulates the onset or progression of a cardiovascular disorder.

17. The method of claim 16, wherein said reference level is derived from a sample derived from said subject.

18. The method of claim 16, wherein said reference level is derived from a database.

19. The method of claim 18, wherein said database comprises test levels of an analyte in a sample derived from a database subject, wherein said database subject is not said test subject.

20. A kit comprising a detection reagent that identifies two or more analytes selected from the group consisting of CVD 1-112.
